(19) United States
(12) Patent Application Publication
(10) Pub. No.: US 2004/0002718 A1
(43) Pub. Date: Jan. 1, 2004

(54) Apparatus and method for placement of surgical fasteners

(76) Inventors: Hugh Trout III, Bethesda, MD (US); Howard Tanner, Logan, UT (US)

Correspondence Address: COLLIER SHANNON SCOTT, PLLC, 3050 K STREET, NW, SUITE 400, WASHINGTON, DC, 20007, US

(21) Appl. No.: 10/417,163

(22) Filed: Apr. 17, 2003

Related U.S. Application Data
(60) Provisional application No. 60/372,792, filed on Apr. 17, 2002.

(51) Int. Cl. A61B 17/10

(52) U.S. Cl. 606/139

(57) ABSTRACT

An embodiment of the present invention relates to an apparatus and method for placement of surgical fasteners. An embodiment of the apparatus comprises an insertion assembly, a handle, and a delivery catheter. The insertion assembly may be a force member, or propulsion assembly, which is integrated within a handle itself attached to a delivery catheter. The apparatus may provide for delivery of at least one fastener through a catheter assembly and its subsequent deployment at a surgical site.

### CROSS-REFERENCE TO RELATED APPLICATIONS

The present invention relates to, and is entitled to the benefit of the earlier filing date and priority of U.S. Provisional Application Serial No. 60/372,792, filed on Apr. 17, 2002.

### FIELD OF THE INVENTION

The present invention relates to methods and apparatus for placement of surgical fasteners. In particular, the methods and apparatus are directed to a handle and an insertion assembly to effect placement of surgical fasteners at a surgical site.

### BACKGROUND OF THE INVENTION

An aneurysm is a ballooning of the wall of an artery resulting from the weakening of the artery due to disease or other conditions. Left untreated, the aneurysm will frequently rupture, resulting in loss of blood through the rupture and death.

Aortic aneurysms are the most common form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in that portion of the abdominal aorta between the renal arteries and the iliac arteries. This portion of the abdominal aorta is particularly susceptible to weakening, resulting in an aortic aneurysm. Such an aneurysm is often located near the iliac arteries. An aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm because no statistical benefit exists in performing such procedures.

Aneurysms in the abdominal aorta are associated with a particularly high mortality rate; accordingly, current medical standards call for urgent operative repair. Abdominal surgery, however, results in substantial stress to the body. Although the mortality rate for an aortic aneurysm is high, there is also considerable mortality and morbidity associated with open surgical intervention to repair an aortic aneurysm. This intervention involves penetrating the abdominal wall to the location of the aneurysm to reinforce or replace the diseased section of the aortic aneurysm. A prosthetic device, typically a synthetic tube graft, is used for this purpose. The graft serves to exclude the aneurysm from the circulatory system, thus relieving pressure and stress on the weakened section of the aorta at the aneurysm.

Repair of an aortic aneurysm by surgical-means is a major operative procedure. Substantial morbidity accompanies the procedure, resulting in a protracted recovery period. Further, the procedure entails a substantial risk of mortality. While surgical intervention may be indicated and the surgery carries attendant risk, certain patients may not be able to tolerate the stress of intra-abdominal surgery. It is, therefore, desirable to reduce the mortality and morbidity associated with intra-abdominal surgical intervention.

In recent years, methods have been developed to attempt to treat an aortic aneurysm without the attendant risks of intra-abdominal surgical intervention. Among them are inventions disclosed and claimed in Kornberg, U.S. Pat. No. 4,562,596 for Aortic Graft, Device and Method for Performing an Intraluminal Abdominal Aortic Aneurysm Repair; Lazarus, U.S. Pat. No. 4,787,899 for Intraluminal Graft Device, System and Method; and Taheri, U.S. Pat. No. 5,042,707 for Intravascular Stapler, and Method of Operating Same

Although in recent years certain techniques have been developed that may reduce the stress, morbidity, and risk of mortality associated with surgical intervention to repair aortic aneurysms, including delivery catheter assemblies, none of the systems that have been developed provide an apparatus for placement of surgical fasteners through a delivery catheter comprising a handle and an insertion assembly comprising an integrated force member or propulsion assembly. An embodiment of the present invention provides an apparatus that can be used by an interventionist outside the body to effectively deploy fasteners for securing a surgical component to tissue, tissue-to-tissue, or surgical component to surgical component. An embodiment of the present invention further provides for minimal intrusiveness during repair of an aortic aneurysm.

Furthermore, an embodiment of the present invention provides a method and apparatus that may be used to occlude or reduce blood flow in certain blood vessels. It is occasionally necessary to occlude or reduce blood flow in certain blood vessels during operative procedures to repair aortic aneurysms. For example, when one or more of the arterial branches of an aneurysm, which are generally the lumbar vessels and the inferior mesenteric artery, are patent and sufficiently large to cause back bleeding when the aneurysm is bypassed with a surgical component, the aneurysm, because of this arterial flow into the aneurysm from the backflow, may still rupture. Normally, if the vessel is large enough, it is relatively easy for an interventionist to insert a wire into the orifice of the vessel and occlude, or close, the vessel with especially designed occluding material. On occasion, however, it is difficult to cannulate the orifice of the vessel and, thus, back bleeding persists. Hence, there is a need for an improved method of occluding a vessel to effect a reduction in blood flow through the vessel during the repair of aortic aneurysms, as well as various other procedures. In particular, if it is recognized that one or more of these aneurysm branches might be a problem after a surgical component is inserted it may be possible to cause occlusion by placing one or more surgical fasteners near the orifice. Accordingly, an embodiment of the present invention provides a method for placing surgical fasteners at a surgical site to occlude or reduce blood flow in a vessel during an operative procedure.

It is therefore an advantage of an embodiment of the present invention to provide an apparatus and method for facilitating the repair of aortic aneurysms. It is another advantage of an embodiment of the present invention to provide an apparatus for placement of surgical fasteners to attach tissue to tissue, a surgical component to a surgical component, or a surgical component to tissue. A further advantage of an embodiment of the present invention is to provide an apparatus and method for placement of surgical fasteners to occlude or reduce blood flow within a vessel. It is yet another advantage of an embodiment of the present invention to provide a method for placement of surgical fasteners at a surgical site.

Additional advantages of embodiments of the invention are set forth, in part, in the description which follows and, in part, will be apparent to one of ordinary skill in the art from the description and/or from the practice of the invention.

### SUMMARY OF THE INVENTION

Responsive to the foregoing challenges, Applicant has developed an innovative apparatus for placement of surgical fasteners, an embodiment may comprise an insertion assembly having a proximal end and a distal end, a handle housing the proximal end of the insertion assembly, and a delivery catheter attached to a distal end of the handle and housing the distal end of the insertion assembly. The apparatus may further comprise an articulation adjustment mechanism for articulating a distal end of the delivery catheter. The insertion apparatus may further comprise at least one fastener, a force member, and/or a propulsion assembly.

An alternative embodiment may comprise a force member having a proximal end and a distal end, and a handle, within which the force member is integrated and which is attached to a delivery catheter. The force member may comprise a pull knob at the proximal end of the member, a piston at the distal end of the member, and an expansile member, connected to, and extending between pull knob and piston. The apparatus may further comprise at least one fastener positioned at the distal end of the force member.

Alternatively, in an embodiment the force member may comprise a force rod extending into the delivery catheter, and a stop mechanism for limiting advancement of the force rod. An embodiment of the apparatus may further comprise at least one fastener positioned at the distal end of the force member.

In an alternative embodiment, the apparatus for placement of surgical fasteners may comprise a propulsion assembly, and a handle, which is connected to a delivery catheter, through which the propulsion assembly passes. The propulsion assembly may comprise an optical fiber having a hollow core and a proximal end and a distal end, wherein the distal end extends within the delivery catheter, a laser may be connected to the proximal end of the optical fiber, an optical assembly having a sealed portal, which provides an opening in the handle, and the optical fiber for introducing at least one fastener into the hollow core of the optical fiber, and an inlet for introducing a compressed gas mixture or fluid stream into the hollow core of the optical fiber for propelling the at least one fastener to a surgical site. The laser may comprise a Holmium-Yag laser. The compressed gas may be selected from the group consisting of carbon dioxide, oxygen, helium or any other suitable gas. The apparatus may further comprise at least one fastener introduced into the hollow core of the optical fiber.

Alternatively, the propulsion assembly may comprise a driver located within the delivery catheter, a piston located within the catheter and between,the driver and at least one distally located fastener, and an inlet for introducing carbon dioxide, oxygen, helium, a fluid stream, or any other suitable gas or liquid, into the assembly for propelling the driver into the piston, thereby effecting placement of the at least one fastener at a surgical site. The compressed gas may be selected from the group consisting of carbon dioxide, oxygen, helium or any other suitable gas.

An embodiment of the present invention is also directed to a method for placement of surgical fasteners, comprising the steps of: gaining access to a surgical site with a delivery catheter; positioning a force member within the delivery catheter; drawing back a pull knob of the force member, thereby extending a an expansile member, such as, but not limited to, a sheathed or unsheathed extension spring, coil, or other expansible material, located in the mid-section of the force member and connecting the pull knob to the piston; and releasing the pull knob, thereby transmitting energy created by the expansile member to a piston and thereafter to at least one fastener for effecting placement of the fastener at a surgical site.

An alternative method for placement of surgical fasteners in accordance with an embodiment of the present invention comprises the steps of: gaining access to a surgical site with a delivery catheter; feeding a force member through the delivery catheter; applying manual force to a force rod of the force member to advance a fastener; prohibiting over advancement of the force member with a stop mechanism; and separating the force member from the fastener, thereby deploying the fastener. A further step may include withdrawing the force member from the surgical site through the delivery catheter.

Another alternative method for placement of surgical fasteners in accordance with an embodiment of the present invention comprises the steps of: gaining access to a surgical site with a delivery catheter; positioning a propulsion assembly within the delivery catheter; activating a laser light source connected to an optical fiber; advancing the optical fiber having a hollow core, thereby creating the treatment specific hole; deactivating the laser; loading at least one fastener into the hollow core of the fiber; propelling the at least one fastener to the surgical site by either a gas mixture or a fluid stream that enters the hollow core through an inlet; and withdrawing the optical fiber from the surgical site, thereby deploying the at least one fastener.

Yet another alternative method for placement of surgical fasteners in accordance with an embodiment of the present invention comprises the steps of: providing a conduit with a delivery catheter that extends between a point of proximal access to a distally positioned surgical site; positioning at least one fastener around, within, or in conjunction with an insertion assembly; forming a treatment specific hole in a surgical component and tissue with the fastener or the insertion assembly; gaining access to the adventitial layer of the vessel wall, facilitating attachment of at least one fastener to an intraluminal side of the surgical component and the adventitial tissue layer; and deploying the at least one fastener by removing the insertion assembly from within, about, or in conjunction with the fastener. The insertion assembly, which may be solid or tubular, may comprise a silica optical fiber, a piezoelectric driven device, such as, but not limited to a Nitinol catheter device, a force member, a propulsion assembly, or any other suitable device. The treatment specific hole may be formed by a Holmium-Yag laser, a piezoelectric driven catheter device, or any other suitable device.

Another alternative method of an embodiment of the present invention for placement of surgical fasteners to occlude a vessel, thereby effecting a reduction in blood flow through the vessel comprises the steps of: gaining access to a surgical site; creating a treatment specific hole in one or more vessel walls at the surgical site; inserting at least one fastener through the treatment specific hole; and deploying the fastener at the surgical site, thereby occluding or partially occluding the vessel.

It is to be understood that both the foregoing general description and the following detailed description are exemplary and explanatory only, and are not restrictive of the invention as claimed. The accompanying drawings, which are incorporated herein by reference, and which constitute a part of this specification, illustrate certain embodiments of the invention and, together with the detailed description, serve to explain the principles of certain embodiments of the present invention.

### BRIEF DESCRIPTION OF THE DRAWINGS

In order to assist the understanding of this invention, reference will now be made to the appended drawings, in which like reference numerals refer to like elements. The drawings are exemplary only, and should not be construed as limiting the invention.

FIG. 1A is a perspective view of an apparatus for placement of surgical fasteners of an embodiment of the present invention, including a force member and a handle into which it is integrated;

FIG. 1B is an illustration of energy being created by the extension of an expansile member located in a force member and its transmittal to a piston in accordance with an embodiment of the present invention;

FIG. 2A is a cross-sectional view of the force member and handle according to an embodiment of the present invention;

FIG. 2B is an exterior view of the integrated force member and handle according to an embodiment of the present invention;

FIG. 2C is a sectional view of the relative positioning of the expansile member, piston, and fastener within an inner sheath of a delivery catheter according to an embodiment of the present invention;

FIG. 3A is a perspective view of an embodiment of the present invention, including a force member and handle into which the member may be integrated.

FIG. 3B is a cross-sectional view of the force member and handle according to an embodiment of the present invention;

FIG. 4A is a cross-sectional view of yet another embodiment of the present invention, including a handle and propulsion assembly;

FIGS. 4B through 4F illustrate the placement of the shaped fastener according to an embodiment of the present invention;

FIGS. 5A through 5D illustrate the occlusion of a vessel according to a method of an embodiment of the present invention; and

FIGS. 6A through 6D illustrate the occlusion of a vessel near an aneurysm according to a method of an embodiment of the present invention.

### DETAILED DESCRIPTION OF EMBODIMENTS OF THE INVENTION

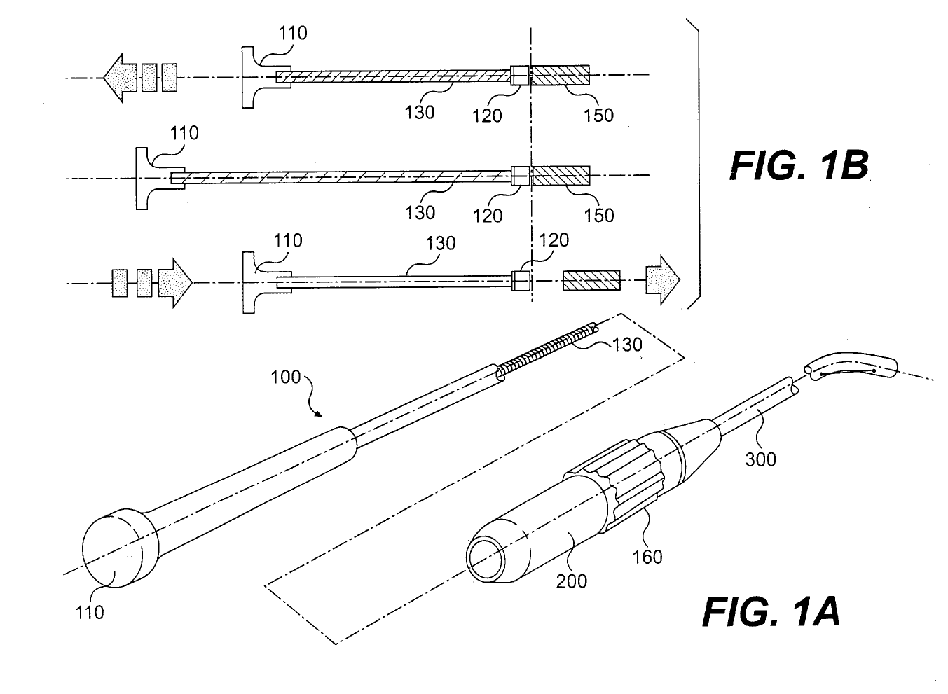

Reference will now be made in detail to embodiments of the present invention, examples of which are illustrated in the accompanying drawings. With reference to FIGS. 1, 2, 3 and 4, the apparatus for placement of surgical fasteners comprises an insertion assembly comprised of a force member 100 or a propulsion assembly 400, a handle 200, and a delivery catheter 300.

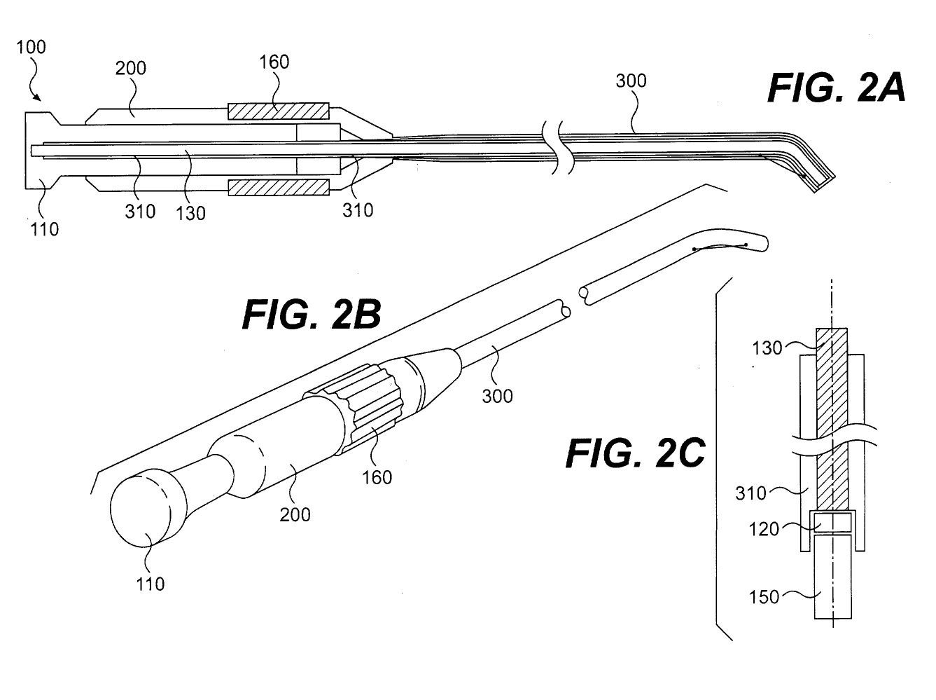

An embodiment of the present invention, as shown in FIGS. 1 and 2 comprises a force member 100, having a proximal end and a distal end, and a handle 200, having a proximal end and a distal end. The proximal end of the force member 100 may be integrated into the handle 200. The distal end of handle 200 is attached to the proximal end of a delivery catheter 300.

In accordance with an embodiment of the present invention, as shown in FIGS. 1 and 2, the force member 100 comprises a pull knob 110 at the proximal end of the force member and a piston 120 at the distal end. An expansile member 130 extends between the pull knob 110 and the piston 120. The expansile member 130 connects the knob 110 and the piston 120. The expansile member 130 may be a spring, coil, or any suitable structure or comprise any suitable material. When the expansile member 130 is extended, energy is created, which can be transmitted axially upon release, to the piston 120 and thereafter to a fastener 150, as shown in FIG. 1B. The force member 100 may be constructed of biocompatible and procedure appropriate materials, including, but not limited to, certain metals such as nitinol or stainless steel, polymeric materials such as nylon, or any other suitable materials. Force member 100 ductile configuration facilitates its insertion into the delivery catheter 300 as well as its angular positioning within the delivery catheter 300.

At least one fastener 150 may be positioned distally with respect to the force member 100. Fasteners may be, but are not limited to, those as disclosed in Tanner, et al., U.S. Pat. No. 5,957,940; Tanner, et al., U.S. Pat. No. 5,972,023; and Tanner, U.S. Pat. No. 5,997,556, which are incorporated herein in their entirety by reference. As depicted in FIG. 2c, the expansile member 130, piston 120, and fastener 150 may be positioned within an inner sheath 310 of the delivery catheter 300. The fastener 150 may be positioned for advancement about, within, or in conjunction with the distal end of the force member 100. For instance, the fastener 150 may be contained in a multi-chambered cylinder radially disposed about the force member 100 as disclosed in Tanner, et al., and U.S. Pat. No. 5,972,023, which is incorporated herein in its entirety by reference. Alternatively, the fastener 150 may be axially aligned for ready insertion into a surgical component and tissue combination, or a tissue-to-tissue combination or a surgical component to surgical component combination, at a surgical site.

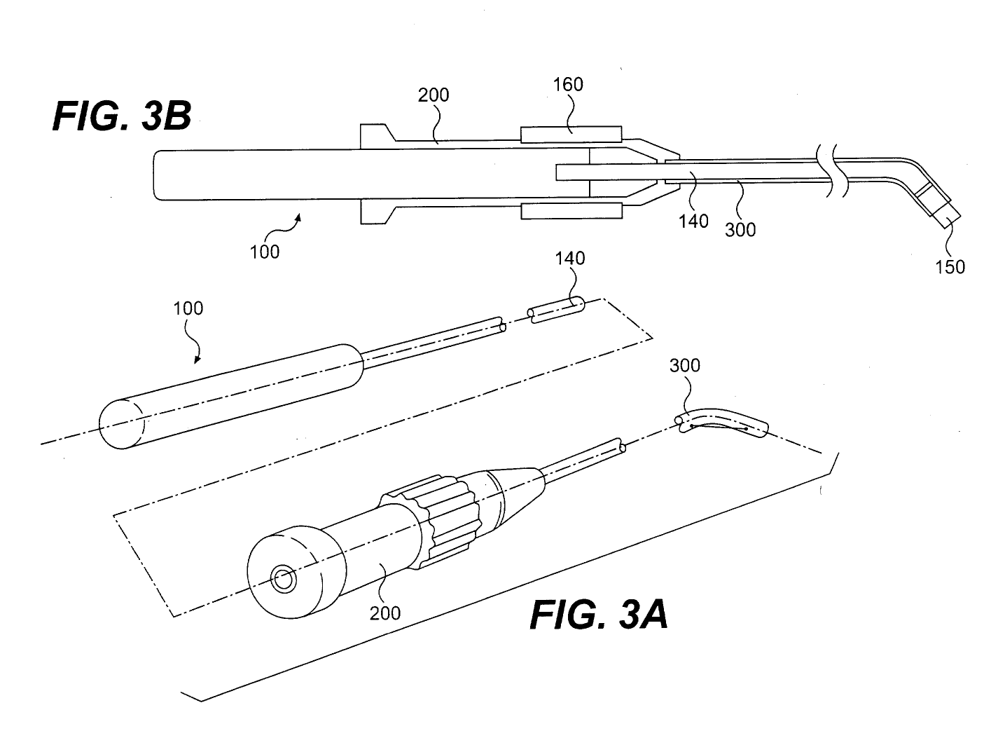

In an alternative embodiment of the present invention, as shown in FIG. 3, the force member 100 comprises a force rod or tube 140, which has a proximal end and a distal end, and a stop or attenuating mechanism. The force member 100 is comprised of any biocompatible and structurally appropriate materials and not limited to those disclosed above. The force rod 140 extends from the interventionist through the handle 200 and delivery catheter 300. At least one fastener 150 is positioned about, within, or in conjunction with the distal end of the force member 100 within the delivery catheter 300. The stop mechanism prevents further/over? advancement of the force rod 140, and thereafter, the fastener 150 within the tissue/surgical component and derivative matrices

Alternatively, according to an embodiment of the present invention, a plurality of fasteners 150 may be positioned end-to-end within the force member 100. In this embodiment, a membrane of reduced cross-section connects the head of one fastener to a tail of an adjacent fastener. When the fastener is appropriately placed at the surgical site, as when penetrating a surgical component and tissue combination, an electric current, heat, piezoelectric energy, or any other suitable means may be applied to the force member and transmitted to separate the most distal fastener from the string of fasteners, thereby facilitating removal of the force member and fastener string from the site and, deployment of the most distal fastener at the surgical site.

The embodiments depicted in FIGS. 1, 2, 3 and 4 may also include an articulation control mechanism 160 located on the handle 200. The mechanism 160 allows for articulation of the distal end of the delivery catheter 300 in preparation for placement of at least one fastener 150 at the surgical site. The mechanism 160 may comprise a knob surrounding the handle 200 for translating rotary into linear motion. This motion pulls or releases a catheter filament to articulate the distal end of the delivery catheter 300. The catheter filament may comprise a braided wire, a single wire, a monofilament, or any other suitable filament, such as, but not limited to, Kevlar®, Spectra®, or any other suitable wire, filament, or material.

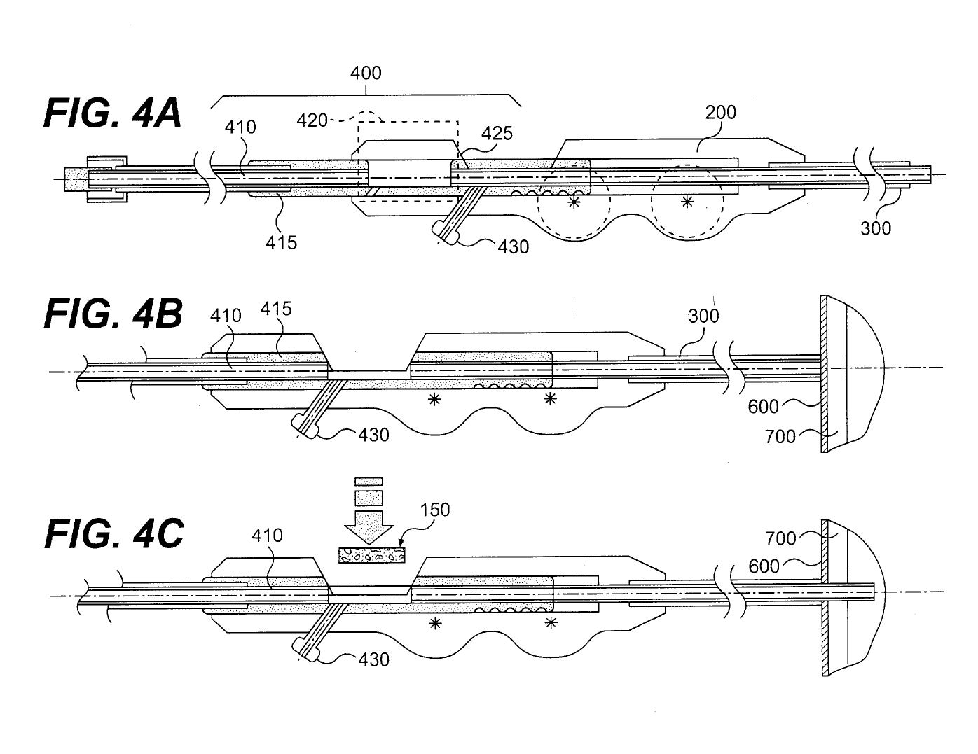

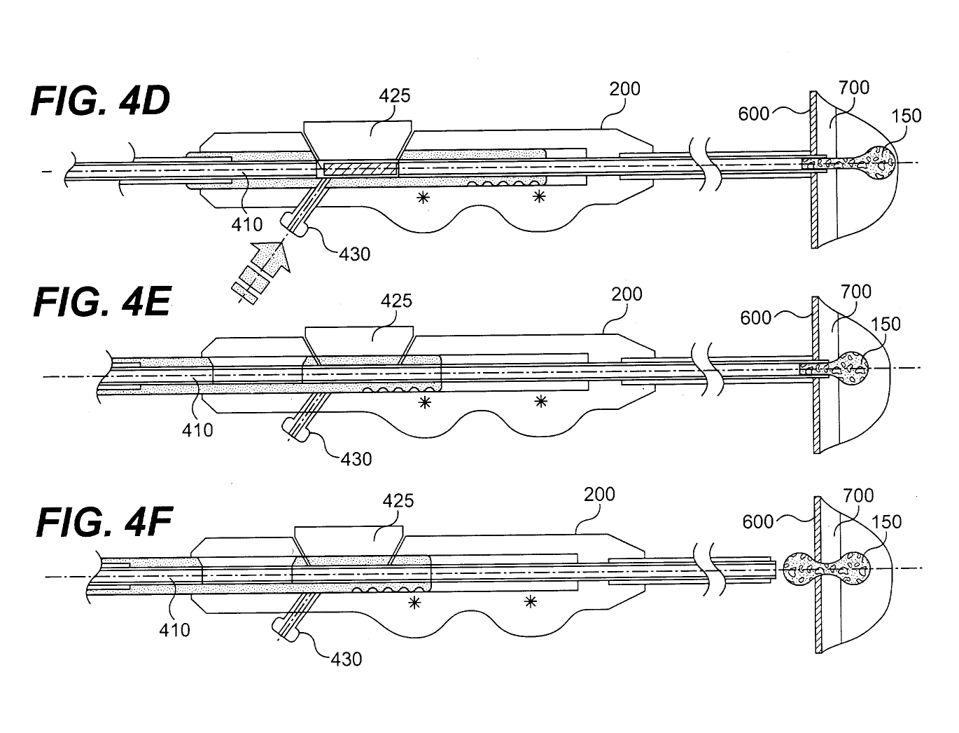

In an alternative embodiment of the present invention, as shown in FIG. 4, the insertion assembly comprises a propulsion assembly 400 and a handle 200. The propulsion assembly 400 passes through the distal end of the handle 200 into a delivery catheter 300, which is connected to the distal end of the handle 200.

In an alternative embodiment, as depicted in FIG. 4, the propulsion assembly 400 includes an optical fiber 410 having a hollow core and proximal and distal ends. The fiber 410 extends distally within the delivery catheter 300 to the surgical site and proximally, through the handle 200. The fiber 410 may have a fiber carrier 415 surrounding it. A laser may be connected to the proximal end of the optical fiber 410. An optical assembly 420 having a portal 425 therein may provide an opening in the handle 200 enabling the introduction of at least one fastener 150 within the hollow core of the fiber 410. An inlet 430 may be included for introducing a propellant into the hollow core of the fiber 410. The propellant propels at least one fastener 150 to the surgical site. The propellant may comprise a gas, gas mixture, fluid, or any other suitable material. The compressed gas may comprise, but is not limited to, carbon dioxide, oxygen, helium, or any other suitable gas. The fluid stream may comprise, but is not limited to, any biocompatible fluid.

In accordance with an embodiment of the present invention, the laser connected to the optical fiber 410 may comprise a Holmium-Yag or similar laser as disclosed in Tanner, et al., and U.S. Pat. No. 5,944,750, which is incorporated herein by reference in its entirety.

According to an alternative embodiment, the at least one fastener 150 introduced into the hollow core of the fiber 410 may be cylindrically shaped. The fastener 150 may be as disclosed previously, and may also be comprised of metal or plastic foam mesh in a highly compressed form, or made of any other suitable material. According to an embodiment of the present invention, the fastener 150 expands or otherwise assumes a secondary configuration upon temperature change, re-hydration, or any other suitable stimulus. This expansion or configuration change may be helpful in holding the fastener 150 in place when implanted at the surgical site.

In an alternative embodiment of the present invention, the propulsion assembly 400 includes a driver and a piston located within a delivery catheter. The piston may be positioned distally with respect to the driver. The piston may be abutting at least one fastener, which is positioned distally within, about, or in conjunction with the delivery catheter for placement at the surgical site. The assembly may also include an inlet for introducing a propellant, such as, but not limited to, a compressed gas or fluid stream into the assembly, such as, carbon dioxide, oxygen, helium, or fluid for propelling the driver into the piston, thereby effecting placement of the fastener at a surgical site. Force created by the propulsion assembly may implant the fastener in a surgical component and tissue combination, or alternatively, a surgical component to surgical component, or a tissue-to-tissue combination.

The operation of an embodiment of the present invention for placement of surgical fasteners will now be described. As shown in FIGS. 1, 2, 3, and 4, an embodiment of the present invention provides access to a surgical site through a delivery catheter, positions an insertion assembly in a handle and the delivery catheter, provides insertion means to deploy at least one fastener at a surgical site. The insertion means may comprise a force member, a propulsion assembly, or any other suitable insertion means.

In accordance with an embodiment shown in FIGS. 1 and 2, access to the surgical site is gained with a delivery catheter 300. The force member 100 of the apparatus is positioned within the delivery catheter 300 and integrated handle 200. As illustrated in FIG. 1B, the pull knob 110 of the force member 100 may be drawn back a defined distance, thereby extending the expansile member 130 that is connected to the pull knob 110 and the piston 120. Movement of piston 120 may be restricted such that withdrawing the pull knob 110 extends the expansile member 130. The extension of the expansile member 130 creates energy within the force member 100. The pull knob 110 may be released, transmitting the energy created to the piston 120 and thereafter to the at least one fastener 150, thereby driving the fastener 150 into position at the surgical site. In particular, the fastener 150 may be implanted into a surgical component and tissue combination for attaching the surgical component to a vessel wall, or into a tissue-to-tissue combination, or surgical component to surgical component combination, or tissue to tissue such that blood flow is reduced or occluded within a vessel.

In accordance with the alternative embodiment shown in FIG. 3, access to the surgical site is gained with a delivery catheter 300. The distal end of the force member 100 may be fed through the delivery catheter 300 to the surgical site. The at least one fastener 150 is positioned at the distal end of the force member 100 for advancement about, within, or in conjunction with the force member 100. Manual force may be applied to the force rod 140 of the force member 100. Force may be applied until a stop mechanism prohibits over advancement of the force member 100. This may be the point when the fastener 150 has been driven through a surgical component and tissue combination to-the tissue's adventitial layer. The force member 100 may be separated from the fastener 150 and withdrawn through the delivery catheter 300, thereby deploying the fastener at a surgical site.

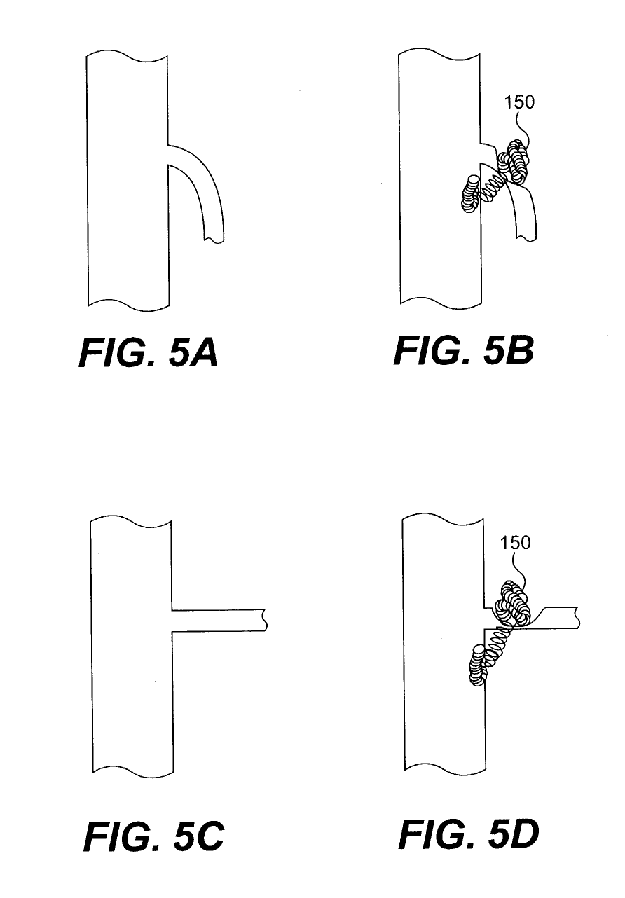

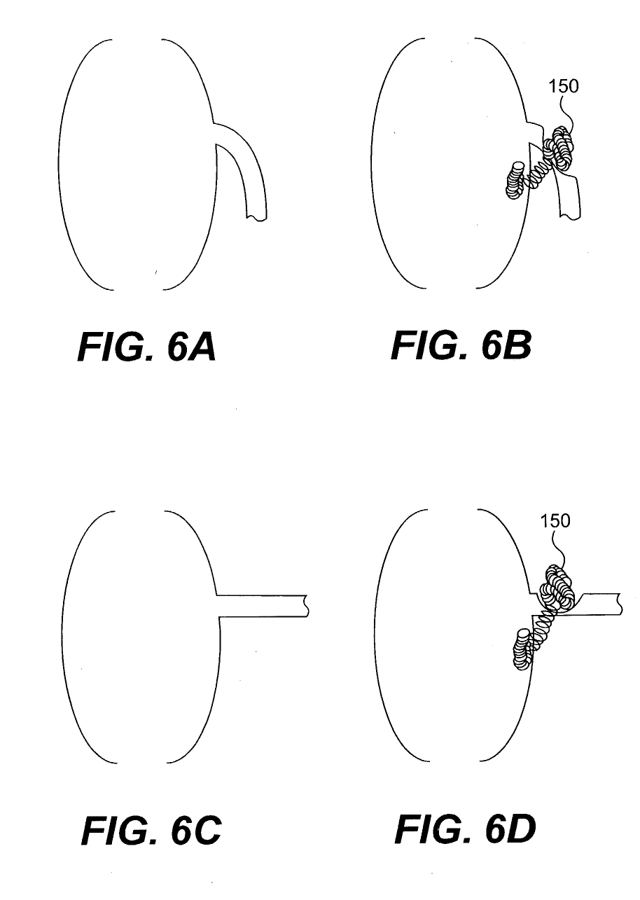

In another method for placement of fasteners, in accordance with an alternative embodiment of the present invention depicted in FIG. 4, access to the surgical site is gained with a delivery catheter 300. A laser connected to the proximal end of the optical fiber 410 may be activated to create a treatment specific hole in a surgical component 600 and tissue 700 combinations. Optical fiber 410 is advanced within the delivery catheter 300 creating and advancing through the treatment specific hole, traversing the surgical component 600 and tissue 700. At this point, the laser may be deactivated. At least one fastener 150 is loaded into a hollow core of the optical fiber 410 through a portal 425 of an optical assembly 420 disposed within handle 200. A propellant, such as, but not limited to, gas mixture or fluid stream may enter the hollow core of the fiber 410 through an inlet 430 and propels the at least one fastener 150 through the hollow core to its placement at the surgical site. The optical fiber 410 may be withdrawn from the treatment specific hole, thereby deploying the fastener 150. The at least one fastener 150 may be deployed to secure a surgical component to a vessel wall, or to another surgical component, or to secure tissue of a vessel wall to tissue of another vessel wall, or tissue to tissue so as to reduce or occlude blood flow within a vessel, as shown in FIGS. 5B, 5D, 6B, and 6D.

In an alternative embodiment of the present invention incorporating fastener 150, deployment of such a fastener will now be described in accordance with FIG. 4. Once the propulsion assembly 400 propels the fastener 150, through a surgical component 600 to the adventitial layer of tissue 700, the temperature is changed or the fastener 150 is re-hydrated or, the fastener 150 assumes a second configuration induced by any suitable mechanism. Enabled by temperature change, re-hydration or, assumption of a second configuration, the distal end of the fastener 150 expands, collapses, twists, or otherwise engages the adventitial tissue layer, as illustrated in FIG. 4D. The optical fiber 410 may be progressively withdrawn from the treatment specific hole, bringing the proximal end of the fastener 150 with it, as shown in FIG. 4E. As the fiber 410 clears the luminal surface of the surgical component 600, the proximal end of the fastener 150 may be deployed and immediately expands, collapses, twists, or assumes its secondary configuration, as disclosed above and as illustrated in FIG. 4F. Both ends of the fastener 150 form head-like details when expanded or in the secondary configuration, holding the surgical component 600 to the tissue 700 in “pop-rivet” fashion. The mid-section of the fastener 150 may also expand circumferentially within the treatment specific hole creating further gripping force, thereby aiding the securement of the fastener 150. The fastener 150 may operate in like manner to secure one or more vessel walls to reduce or occlude blood flow within a vessel.

In accordance with another embodiment of the method for placement of fasteners of an embodiment of the present invention, a treatment conduit may be provided by a delivery catheter or a delivery catheter further comprising an optical fiber having a hollow core. The conduit extends between a point of proximal access, such as a femoral, groin, axillary, or brachial artery, to a distally positioned surgical site. At least one fastener is positioned around, within, or in conjunction with an insertion assembly, which is fed to the surgical site through a delivery catheter. A treatment specific hole is formed through a surgical component and adjacent tissue with the insertion assembly, such as by laser energy transmission. Intraluminal access to an adventitial tissue layer may be gained, facilitating attachment of at least one fastener to both the intraluminal side of a surgical component and the adventitial tissue layer. The at least one fastener may be deployed by removing the insertion assembly from within, about, or in conjunction with the fastener. In accordance with this method, the insertion assembly may comprise a solid or a hollow tubular profile. In an embodiment, the insertion assembly may further comprise a silica optical fiber or piezoelectric-driven Nitinol catheter device, a force member, or a propulsion assembly. The treatment specific hole may be formed by a Holmium-Yag laser, a piezoelectric device, the fastener itself, or any other suitable device. Alternatively, the treatment specific hole may be created in one or more vessel walls for attachment of at least one fastener thereto, for occluding or reducing blood flow within a vessel.

Accordingly, an embodiment of the present invention is directed to a method for reducing or occluding blood flow within a vessel, as depicted in FIGS. 5A through 5D and FIGS. 6A through 6D, comprising the steps of: gaining access to a surgical site; inserting at least one and deploying a fastener at the site, thereby occluding or reducing the blood flow within the vessel. The fastener may be inserted near the orifice of the vessel at a positive angle to the vessel to be occluded. A vessel adjacent an aneurysm may be occluded to prevent back bleeding, as depicted in FIGS. 6A through 6D, or any vessel, may be occluded for various medical reasons, as shown in FIGS. 5A through 5D.

It will be apparent to those skilled in the art that variations and modifications of embodiments of the present invention can be made without departing from the scope or spirit of the invention. For example, an embodiment of the present invention is not limited to securing a surgical component and/or surgical device, which may be a graft, to a vessel wall. Rather, it is contemplated that an embodiment of the present invention may be used in connection with securing a vessel to another vessel, tissue-to-tissue, surgical components to surgical components, and any variations thereof. In addition, the methods disclosed above are not limited to use with the fastener disclosed in this application. It is well within the scope of an embodiment of the present invention that embodiments of the methods and apparatus herein can be used with the fasteners disclosed in Tanner, et al., and U.S. Pat. No. 5,957,940; Tanner, et al., U.S. Pat. No. 5,972,023; and Tanner, U.S. Pat. No. 5,997,556, which are incorporated herein by reference. Furthermore, the method of reducing blood flow within a vessel is not limited to the fastener placement methods disclosed herein. It is envisioned by Applicant that the method of using fasteners to reduce blood flow may be achieved by any placement or insertion method. Thus, it is intended that an embodiment of the present invention cover all such modifications and variations of the invention, provided they come within the scope of the appended claims and their equivalents.

What is claimed is:

1. An apparatus for placement of surgical fasteners, comprising:
an insertion assembly having a proximal end and a distal end;
a handle housing the proximal end of the insertion assembly; and
a delivery catheter attached to a distal end of the handle and housing the distal end of the insertion assembly.

2. The apparatus according to claim 1, wherein the insertion apparatus further comprises at least one fastener.

3. The apparatus according to claim 1, wherein the insertion apparatus further comprises a force member.

4. The apparatus according to claim 1, wherein the insertion apparatus further comprises a propulsion assembly.

5. The apparatus according to claim 1, further comprising an articulation adjustment mechanism for articulating a distal end of the delivery catheter.

6. The apparatus according to claim 3, wherein the force member further comprises:
a pull knob at the proximal end of the member;
a piston at the distal end of the member; and
an expansile member with a first end connected to a distal end of the pull knob and a second end connected to a proximal end of the piston.

7. The apparatus according to claim 3, wherein the force member further comprises:
a force rod extending into the delivery catheter; and
a stop mechanism for controlling advancement of the force rod into the delivery catheter.

8. The apparatus according to claim 3, further comprising at least one fastener positioned at the distal end of the force member.

9. The apparatus according to claim 4, wherein the propulsion assembly further comprises:
an optical fiber having a hollow core, a proximal end, and a distal end, wherein the distal end extends into the delivery catheter;
an optical assembly having a portal therein, which enables the introduction of at least one fastener into the hollow core of the optical fiber; and
an inlet for introducing a propelling means into the hollow core of the optical fiber for propelling the at least one fastener to a surgical site.

10. An apparatus for placement of surgical fasteners, comprising:
a force member having a proximal end and a distal end; and
a handle, within which the force member is integrated and wherein a distal end of the handle is attached to a delivery catheter.

11. An apparatus for placement of surgical fasteners, comprising:
a propulsion assembly; and
a handle, within which the propulsion assembly is integrated, and wherein a distal end of the handle is connected to a delivery catheter.

12. The apparatus according to claim 11, wherein the propulsion assembly comprises:
an optical fiber having a hollow core, a proximal end, and a distal end, wherein the distal end extends into the delivery catheter;
an optical assembly having a portal therein, which enables the introduction of at least one fastener into the hollow core of the optical fiber; and
an inlet for introducing a propellant into the hollow core of the optical fiber for propelling the at least one fastener to a surgical site.

13. The apparatus according to claim 12, wherein the propellant is a gas.

14. The apparatus according to claim 12, wherein the propellant is a liquid.

15. The apparatus according to claim 12, wherein the propellant is selected from the group consisting of carbon dioxide, oxygen, and helium.

16. The apparatus according to claim 12, wherein the proximal end of the optical fiber is attached to a laser.

17. The apparatus according to claim 16, wherein the laser is Holmium-Yag.

18. The apparatus according to claim 12, further comprising at least one fastener disposed within the hollow core of the optical fiber.

19. The apparatus according to claim 18, wherein the fastener comprises metal or plastic foam mesh in a highly compressed form.

20. A method for placement of surgical fasteners, comprising the steps of:
accessing a surgical site with a delivery catheter;
positioning a force member within the delivery catheter;
drawing back a pull knob of the force member, thereby extending an expansible member disposed in the mid-section of the force member; and
releasing the pull knob, thereby transmitting energy stored in the expansible member to a piston and thereafter to at least one fastener at a surgical site.

21. A method for placement of surgical fasteners, comprising the steps of:
accessing a surgical site with a delivery catheter;
feeding a force member through the delivery catheter;
applying manual force to a force rod of the force member to advance at least one fastener;
separating the force member from the at least one fastener, thereby deploying the fastener; and
withdrawing the force member through the delivery catheter from the surgical site.

22. A method for placement of surgical fasteners, comprising the steps of:
accessing a surgical site with a delivery catheter;
positioning a propulsion assembly within the delivery catheter;
activating a laser connected to an optical fiber having a hollow core;
advancing the optical fiber, thereby creating a treatment specific hole at the surgical site;
deactivating the laser;
loading at least one fastener into the hollow core of the fiber;
propelling the at least one fastener to the surgical site by a propellant that enters the hollow core through an inlet; and
withdrawing the optical fiber from the surgical site, thereby deploying the at least one fastener at the surgical site.

23. A method for placement of surgical fasteners, comprising the steps of:
providing a delivery catheter extending between a point of proximal access to a distally positioned surgical site;
positioning a portion of the insertion assembly containing at least one fastening means within the delivery catheter;
advancing the delivery catheter and insertion assembly housed therein to a surgical site;
forming a treatment specific hole in a surgical component and tissue by the advancement of a portion of the insertion assembly;
inserting at least one fastener to an intraluminal side of the surgical component and the adventitial tissue layer; and
deploying the at least one fastener by removing the insertion assembly.

24. The method according to claim 23, wherein the insertion assembly comprises a hollow optical fiber.

25. The method according to claim 23, wherein the insertion assembly comprises a piezoelectric-driven, Nitinol catheter.

26. The method according to claim 23, wherein the insertion assembly comprises a force member.

27. The method according to claim 23, wherein the insertion assembly comprises a propulsion assembly.

28. A method for placement of surgical fasteners to occlude a vessel, thereby effecting a reduction in blood flow through the vessel, the method comprising the steps of:
gaining access to a surgical site;
creating a treatment specific hole in one or more vessel walls at the surgical site;
inserting at least one fastener through the treatment specific hole; and
deploying the fastener within the material matrix at the surgical site.
